(19) United States
(12) Patent Application Publication
(10) Pub. No.: US 2009/0192155 A1
(43) Pub. Date: Jul. 30, 2009

(54) Identification of Compounds Suitable for Treating Ad

(76) Inventors: Ian Churcher, Herts (GB); Peter Hunt, Sussex (GB); Carmel Nanthakumar, Middlesex (GB); Peter Simpson, Macclesfield (GB)

Correspondence Address: MERCK AND CO., INC, P O BOX 2000, RAHWAY, NJ, 07065-0907, US

(21) Appl. No.: 12/223,360

(22) PCT Filed: Jan. 30, 2007
(86) PCT No.: PCT/GB2007/050046

§ 371 Date: Dec. 16, 2008

(30) Foreign Application Priority Data
Feb. 3, 2006 (GB) 0602176.0

(51) Int. Cl. A61K 31/5377; C12Q 1/48; A61K 31/496; A61P 25/28

(52) U.S. Cl. 514/234.5; 435/15; 514/254.06

(57) ABSTRACT

The invention provides a method of screening for compounds which inhibit the hyperphosphorylation of tau, and hence are suitable for treating AD and related conditions.

This invention relates to methods for identifying materials capable of inhibiting the hyperphosphorylation of tau protein. The invention further relates to compounds identified by said methods and their use in the treatment or prevention of neurodegenerative diseases such as Alzheimer's disease.

Alzheimer's disease (AD) is the most common cause of dementia in the elderly and is characterised by a decline in cognitive function that progresses slowly and results in symptoms such as memory loss and disorientation. Death occurs, on average, 9 years after diagnosis. The incidence of AD increases with age, so that while about 5% of people over the age of 70 are sufferers, this figure increases to 20% of those over 80 years old.

Existing treatments exclusively target the primary symptoms of AD. Diseased neurons may release insufficient or excessive amounts of particular neurotransmitters, and so current drugs are aimed at increasing neurotransmitter levels or at reducing the stimulation of nerve cells by neurotransmitters. Although these drugs provide some improvement in the symptoms of AD, they fail to address the underlying cause of the disease.

The classic clinical and neuropathological features of AD, first described in 1907, consist of senile or neuritic plaques and tangled bundles of fibers (neurofibrillary tangles) [Verdile, G., et al, Pharm. Res. 50:397-409 (2004)]. In addition, there is a severe loss of neurons in the hippocampus and the cerebral cortex. Neuritic plaques are extracellular lesions, consisting mainly of deposits of β-amyloid peptide (Aβ), surrounded by dystrophic (swollen, damaged and degenerating) neurites and glial cells activated by inflammatory processes. In contrast, neurofibrillary tangles (NFTs) are intracellular clusters composed of a hyperphosphorylated form of the protein tau, which are found extensively in the brain (e.g. mainly in cortex and hippocampus in AD). Tau is a soluble cytoplasmic protein which has a role in microtubule stabilisation. Excessive phosphorylation of this protein renders it insoluble and leads to its aggregation into paired helical filaments, which in turn form NFTs.

The amyloid cascade hypothesis proposes that abnormal accumulation of Aβ peptides, particularly Aβ42, initiates a cascade of events leading to the classical symptoms of AD and ultimately, to the death of the patient. Early onset familial AD (FAD), which has all the neuropathological and clinical features of AD, is caused predominantly by mutations in βAPP or presenilin, which are key components in the production of Aβ [Mudher, A., Lovestone, 5. (2002) Trends Neurosci. 25:22-26]. In contrast, no mutations in the tau gene have yet been found to cause FAD, suggesting that changes in βAPP processing represent an upstream event in pathogenesis. However, whilst transgenic mice bearing the FAD mutations show an increase in Aβ42 levels and numbers of neuritic plaques, they fail to show significant neuronal loss, and exhibit minimal tau phosphorylation and no tangle formation, suggesting that another factor is missing which links the two pathologies. Furthermore, cultured hippocampal neurons from tau-knockout mice do not develop neuronal degeneration upon Aβ administration, but if the tau gene is re-expressed, the neurotoxic effect of Aβ is restored [Rapoport, M., et al (2002) Proc. Natl. Acad. Sci. USA 99:6364-6369]. Taken together, these data strongly suggest that dysregulation of tau function is a necessary step in the cascade of Alzheimer's disease pathology leading ultimately to neuronal death.

Furthermore, tau mutations and NFTs are found in other dementias in which Aβ pathology is absent, such as frontotemporal dementia, Pick's disease and parkinsonism linked to chromosome 17 (FTDP-17) [Mizutani, T, (1999) Rinsho Shikeigaku 39: 1262-1263]. Also, in AD the degree of dementia correlates more closely to the frequency of NFTs than to the frequency of senile plaques [Arriagada, P. V., et al (1992) Neurology 42:631-639], while significant numbers of amyloid plaques are often found in the brains of non-demented elderly people, suggesting that amyloid pathology on its own is not sufficient to cause dementia.

For these reasons, normalisation of tau function (in particular prevention of hyperphosphorylation) is seen as a desirable therapeutic goal for the treatment of AD and other dementing conditions.

Tau is a 352-441 amino acid protein encoded by the Mapt (Microtubule-associated protein tau) gene which is widely expressed in the central nervous system (CNS) with localisation primarily in axons [Binder et al J. Cell Biol. 1985, 101(4), 1371-1378]. The major function of tau is regulation of the stability of microtubules (MTs), intracellular structural components comprised of tubulin dimers which are integral in regulating many essential cellular processes such as axonal transport and elongation as well as generation of cell polarity and shape. Tau binding to tubulin is a key factor in determining the rates of polymerisation/depolymerisation (termed dynamic instability) of MTs, and tau is therefore key to the regulation of many essential cellular processes [see, for example, Butner, K. A., Kirschner, M. W, (1991) J. Cell. Biol. 115: 717-730].

Tau is a basic protein with numerous serine and threonine residues, many of which are susceptible to phosphorylation. While normal tau has two to three phosphorylated amino acid residues, hyperphosphorylated tau found in AD and other tauopathies typically has eight or nine phosphorylated residues. A variety of kinases promote phosphorylation of these sites, including proline-directed kinases such as glycogen synthase kinase 3β (GSK3β) and cyclin dependent kinase 5 (cdk5), and non-proline-directed kinases such as protein kinase A (PKA) and calmodulin kinase II (CaMKII), which phosphorylate tau at Lys-(Ile/Cys)-Gly-Ser sequences, also known as KXGS motifs. One KXGS motif is found in each of the MT binding repeats. Phosphorylation at these sites is important for the regulation of tau-MT binding and while the degree of phosphorylation is normally low, it has been shown to be increased in brain tissue from AD patients. Phosphorylation of one particular residue within the KXGS motifs, Ser-262 has been shown to be elevated in tau protein extracted from the NFTs in AD [Hasegawa, M. et al (1992) J. Biol. Chem. 267:17047-17054] and phosphorylation at this site also appears to dramatically reduce MT binding [Biernat, J. et al. (1993) Neuron 11: 153-163].

There is therefore considerable interest in the use of kinase inhibitors to control the hyperphosphorylation of tau and hence (potentially) to treat or prevent AD and other tauopathies. A particularly interesting target is the microtubule affinity regulating kinase (MARK), since evidence exists that MARK acts as a “master kinase” in that it phosphorylates tau at Ser-262 and thereby primes tau for further phosphorylation by other kinases [Drewes, G, (2004). Trends Biochem. Sci 29:548-555].

The present invention concerns an alternative means of controlling the hyperphosphorylation of tau, with a view to treating or preventing AD and other tauopathies, involving inhibition of brain serine/threonine kinase. Brain serine/threonine kinase (BRSK, also known as brain-specific kinase) is a kinase expressed in the human brain in two isoforms (BRSK1 and BRSK2) and has not hitherto been suggested as a druggable target in the control of AD or related conditions.

Therefore, according to the invention there is provided a screening method for selecting compounds suitable for use in the treatment or prevention of Alzheimer's disease or other condition involving abnormal phosphorylation of tau, said method comprising the steps of:

(i) incubating BRSK with a peptide substrate in the presence of ATP and a test compound, said peptide substrate comprising a phosphorylatable serine residue, under conditions compatible with phosphorylation of said serine residue;

(ii) measuring the degree to which the peptide substrate has become phosphorylated at said serine residue; and

(iii) comparing the result obtained in step (ii) with that obtained from a corresponding blank incubation carried out in the absence of a test compound.

The invention further provides the use of a BRSK inhibitor for the manufacture of a medicament for treatment or prevention of Alzheimer's disease or other condition involving abnormal phosphorylation of tau.

The invention further provides a method of treating or preventing Alzheimer's disease or other condition involving abnormal phosphorylation of tau comprising administering to a patient in need thereof a therapeutically-effective dose of a BRSK inhibitor.

As used herein, the term “BRSK inhibitor” refers to a compound which, when tested in the screening method of the invention, produces a lowering of the degree of phosphorylation in comparison with that obtained from the blank incubation.

The BRSK used in the above screening method is typically obtained via recombinant expression from host cells, and is available from Upstate or the Dundee Kinase Consortium. Either of the two isoforms BRSK1 and BRSK2 may be used.

The present inventors have ascertained that the kinase domains of BRSK1 and BRSK2 have a high degree of homology with the corresponding domains of the MARK isoforms MARK1-4, and it is hypothesised that BRSK acts as a master kinase for the phosphorylation of tau in a similar manner to MARK. In support of this hypothesis, the inventors have further ascertained that BRSK1 and BRSK2 are human homologs of the murine kinases SAD-A and SAD-B which have been shown to phosphorylate tau at Ser-262 [Kishi et al (2005), Science, 307:929-932], and have themselves demonstrated that BRSK1 and BRSK2 phosphorylate tau at Ser-262.

Inhibitors of BRSK identified by the above route may be further screened for activity against MARK, with a view to identifying compounds that are selectively active against BRSK, or which are active against both enzymes. Both types of compound are considered useful, for the reasons discussed below.

Whereas MARK is widely expressed within the CNS and other tissues, expression of BRSK is restricted to the brain. Hence, inhibitors which are selective for BRSK over MARK may inhibit phosphorylation of tau in a neuronal-specific manner and with a reduced propensity for unwanted peripheral effects. On the other hand, compounds active against both BRSK and MARK may provide a more potent inhibition of tau phosphorylation, as there is evidence that BRSK and MARK phosphorylate tau by distinct processes. In the case of MARK, phosphorylation involves binding of 14-3-3 protein, whereas phosphorylation by BRSK does not. Hence, BRSK/MARK dual inhibitors offer the prospect of interrupting two separate phosphorylation pathways.

Inhibitors of BRSK identified by the above method are preferably counterscreened against other kinases, such as Cdk-5, PKA, GSK3β and CaMKII, with a view to identifying compounds which are selective for BRSK, as these are less likely to give rise to unwanted side effects when used for the treatment of AD or other tauopathies.

In the screening method of the invention, the peptide substrate may, in principle, be any peptide comprising a suitable serine residue, including tau itself or a fragment thereof which includes the Ser-262 site. A preferred substrate is Cdc25C peptide, suitably labelled versions of which are available commercially. Cdc25C peptide is a fragment of full-length Cdc25C protein containing the Ser-216 site, and the inventors have established that BRSK phosphorylates Cdc25C at said Ser-216 site.

In the screening method described above, measurement of the degree of phosphorylation may involve any of the standard methods known to those skilled in the art. Very suitably, the peptide substrate used in step (i) is labelled with a fluorescent group and changes in its fluorescent properties are used as a measure of the degree of phosphorylation. Thus, in one embodiment, the peptide substrate is labelled with a fluorescent group and step (ii) comprises binding of the phosphorylated peptide to a metal coordination complex which induces polarisation of the fluorescence of the fluorescent group, the degree of phosphorylation being indicated by the degree of fluorescence polarisation. A suitable fluorescent labelling group is fluorescein. Suitably-labelled Cdc25C peptide is available commercially from Molecular Devices Corp., together with suitable metal complexes and other reagents, under the trademark IMAP®.

In an alternative embodiment, the peptide substrate is labelled with a fluorescent acceptor group, step (ii) comprises binding of the phosphorylated peptide to an antibody labelled with a fluorescent donor group, and the extent of phosphorylation is measured by fluorescence resonance energy transfer (FRET) analysis (i.e. by monitoring the fluorescence of the acceptor group in response to irradiation at the excitation wavelength of the donor group). Very suitably, the fluorescence of both the acceptor group and the donor group are measured in response to irradiation at the excitation wavelength of the donor group, and the ratio of the two used as a measure of the degree of phosphorylation. Preferably, the fluorescence of the acceptor group is measured on a time-resolved basis so as to distinguish the longer-duration fluorescence arising from energy transfer from the shorter-duration fluorescence caused by direct excitation, in the technique known as homogeneous time-resolved fluorescence (HTRF) analysis. Suitable fluorescent donor and acceptor labelling reagents are available commercially, e.g. from Cisbio. The donor is typically a Eu³⁺ cryptate complex attached to an antibody for the phosphorylated serine site on the substrate. The acceptor is suitably allophycocyanin, e.g. in streptavidin-conjugated form as supplied by Cisbio under the trademarks Streptavidin-XL665 and Streptavidin-XL^{ent!}, which is easily linked to a biotinylated peptide substrate. A preferred substrate for use in this embodiment of the invention is therefore biotinylated Cdc25C, which is available commercially from Cell Signalling Technologies. For a review of HTRF, see Kolb A, Burke J, Mathis G., A Homogeneous, Time-Resolved Fluorescence Method for Drug Discovery in: Devlin J P, Ed. High Throughput Screening, the Discovery of Bioactive Substances. New York: Marcel Dekker, Inc. 1997. 345-60.

Counterscreens against other kinases (e.g. MARK, Cdk-5 and PKA) may be carried out by analogous methods, substituting the appropriate kinase for BRSK.

In one embodiment of the invention, a selective BRSK inhibitor is used in the treatment or prevention of Alzheimer's disease or other tauopathy, or in the manufacture of a medicament for treatment or prevention of Alzheimer's disease or other tauopathy. In another embodiment of the invention, a compound capable of inhibiting both BRSK and MARK is used for these purposes.

BRSK inhibitors identified by the method of the invention include the compounds of formula 5:

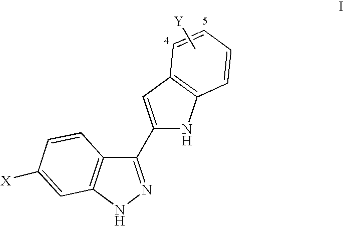

and pharmaceutically acceptable salts thereof; wherein Y is attached at the 4- or 5-position of the indole ring and X and Y are as indicated in the following table:

| Ex. | X | Y | Y-posn. |
|---|---|---|---|
| 1 | CN | 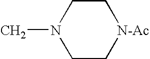 | 5 |
| 2 | CN | 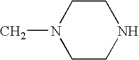 | 5 |
| 3 | CN | CH₂NHCH₂CH₂NH₂ | 5 |
| 4 | CN | 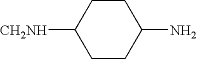 | 5 |
| 5 | CN | 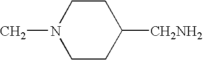 | 5 |
| 6 | CN | 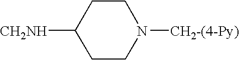 | 4 |
| 7 | CN | 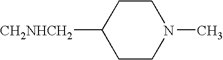 | 4 |
| 8 | CN | (morpholin-4-yl)methyl | 5 |
| 9 | CN | CH₂NHCH₂CH₂—S-t-butyl | 4 |
| 10 | Cl | 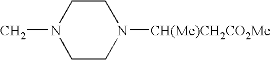 | 4 |
| 11 | 1-H-tetrazol-5-yl | 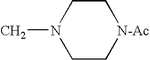 | 5 |
| 12 | 1-H-tetrazol-5-yl | 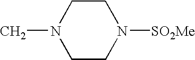 | 5 |
| 13 | CO₂Me | (morpholin-4-yl)methyl | 5 |
| 14 | CONHMe | (morpholin-4-yl)methyl | 5 |
| 15 | 4,5-dihydro-oxazol-2-yl | (morpholin-4-yl)methyl | 5 |

Ac = acetyl

In a further embodiment, the invention provides the use of a compound of formula I as defined above or a pharmaceutically acceptable salt or hydrate thereof for the manufacture of a medicament for treatment of Alzheimer's disease or other tauopathy.

The BRSK inhibitors are suitably administered to patients in the form a pharmaceutical composition comprising the active ingredient (e.g. a compound of formula I or pharmaceutically acceptable salt thereof) and a pharmaceutically acceptable carrier.

Preferably these compositions are in unit dosage forms such as tablets, pills, capsules, powders, granules, sterile parenteral solutions or suspensions, metered aerosol or liquid sprays, drops, ampoules, transdermal patches, auto-injector devices or suppositories; for oral, parenteral, intranasal, sublingual or rectal administration, or for administration by inhalation or insufflation. The principal active ingredient typically is mixed with a pharmaceutical carrier, e.g. conventional tableting ingredients such as corn starch, lactose, sucrose, sorbitol, talc, stearic acid, magnesium stearate and dicalcium phosphate, or gums, dispersing agents, suspending agents or surfactants such as sorbitan monooleate and polyethylene glycol, and other pharmaceutical diluents, e.g. water, to form a homogeneous preformulation composition containing a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This preformulation composition is then subdivided into unit dosage forms of the type described above containing from 0.1 to about 500 mg of the active ingredient of the present invention. Typical unit dosage forms contain from 1 to 100 mg, for example 1, 2, 5, 10, 25, 50 or 100 mg, of the active ingredient. Tablets or pills of the composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compositions useful in the present invention may be incorporated for administration orally or by injection include aqueous solutions, liquid- or gel-filled capsules, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, poly(ethylene glycol), poly(vinylpyrrolidone) or gelatin.

In one embodiment of the invention, the BRSK inhibitor is administered to a patient suffering from AD, FTDP-17, Pick's disease or frontotemporal dementia, preferably AD.

In an alternative embodiment of the invention, the BRSK inhibitor is administered to a patient suffering from mild cognitive impairment or age-related cognitive decline. A favourable outcome of such treatment is prevention or delay of the onset of AD. Age-related cognitive decline and mild cognitive impairment (MCI) are conditions in which a memory deficit is present, but other diagnostic criteria for dementia are absent (Santacruz and Swagerty, American Family Physician, 63 (2001), 703-13). (See also “The ICD-10 Classification of Mental and Behavioural Disorders”, Geneva: World Health Organisation, 1992, 64-5). As used herein, “age-related cognitive decline” implies a decline of at least six months' duration in at least one of: memory and learning; attention and concentration; thinking; language; and visuospatial functioning and a score of more than one standard deviation below the norm on standardized neuropsychologic testing such as the MMSE. In particular, there may be a progressive decline in memory. In the more severe condition MCI, the degree of memory impairment is outside the range considered normal for the age of the patient but AD is not present. The differential diagnosis of MCI and mild AD is described by Petersen et al., Arch. Neurol., 56 (1999), 303-8. Further information on the differential diagnosis of MCI is provided by Knopman et al, Mayo Clinic Proceedings, 78 (2003), 1290-1308. In a study of elderly subjects, Tuokko et al (Arch, Neurol., 60 (2003) 577-82) found that those exhibiting MCI at the outset had a three-fold increased risk of developing dementia within 5 years.

Grundman et al (J. Mol. Neurosci., 19 (2002), 23-28) report that lower baseline hippocampal volume in MCI patients is a prognostic indicator for subsequent AD. Similarly, Andreasen et al (Acta Neurol. Scand, 107 (2003) 47-51) report that high CSF levels of total tau, high CSF levels of phospho-tau and lowered CSF levels of Aβ42 are all associated with increased risk of progression from MCI to AD.

Within this embodiment, the BRSK inhibitor is advantageously administered to patients who suffer impaired memory function but do not exhibit symptoms of dementia. Such impairment of memory function typically is not attributable to systemic or cerebral disease, such as stroke or metabolic disorders caused by pituitary dysfunction. Such patients may be in particular people aged 55 or over, especially people aged 60 or over, and preferably people aged 65 or over. Such patients may have normal patterns and levels of growth hormone secretion for their age. However, such patients may possess one or more additional risk factors for developing Alzheimer's disease. Such factors include a family history of the disease; a genetic predisposition to the disease; elevated serum cholesterol; and adult-onset diabetes mellitus.

In a particular embodiment of the invention, the BRSK inhibitor is administered to a patient suffering from age-related cognitive decline or MCI who additionally possesses one or more risk factors for developing AD selected from: a family history of the disease; a genetic predisposition to the disease; elevated serum cholesterol; adult-onset diabetes mellitus; elevated baseline hippocampal volume; elevated CSF levels of total tau; elevated CSF levels of phospho-tau; and lowered CSF levels of Aβ(1-42).

A genetic predisposition (especially towards early onset AD) can arise from point mutations in one or more of a number of genes, including the APP, presenilin-1 and presenilin-2 genes. Also, subjects who are homozygous for the ε4 isoform of the apolipoprotein E gene are at greater risk of developing AD.

The patient's degree of cognitive decline or impairment is advantageously assessed at regular intervals before, during and/or after a course of treatment in accordance with the invention, so that changes therein may be detected, e.g. the slowing or halting of cognitive decline. A variety of neuropsychological tests are known in the art for this purpose, such as the Mini-Mental State Examination (MMSE) with norms adjusted for age and education (Folstein et al., J. Psych. Res., 12 (1975), 196-198, Anthony et al., Psychological Med., 12 (1982), 397-408; Cockrell et al., Psychopharmacology, 24 (1988), 689-692; Crum et al., J. Am. Med. Assoc'n. 18 (1993), 2386-2391). The MMSE is a brief, quantitative measure of cognitive status in adults. It can be used to screen for cognitive decline or impairment, to estimate the severity of cognitive decline or impairment at a given point in time, to follow the course of cognitive changes in an individual over time, and to document an individual's response to treatment. Another suitable test is the Alzheimer Disease Assessment Scale (ADAS), in particular the cognitive element thereof (ADAS-cog) (See Rosen et al., Am. J. Psychiatry, 141 (1984), 1356-64).

For treating or preventing Alzheimer's disease, a suitable dosage level is about 0.01 to 250 mg/kg per day, preferably about 0.01 to 100 mg/kg per day, and more preferably about 0.05 to 50 mg/kg of body weight per day, of the active compound. The compounds may be administered on a regimen of 1 to 4 times per day. In some cases, however, a dosage outside these limits may be used.

The BRSK inhibitor optionally may be administered in combination with one or more additional compounds known to be useful in the treatment or prevention of AD or the symptoms thereof. Such additional compounds thus include cognition-enhancing drugs such as acetylcholinesterase inhibitors (e.g. donepezil and galanthamine), NMDA antagonists (e.g. memantine) or PDE4 inhibitors (e.g. Ariflo™ and the classes of compounds disclosed in WO 03/018579, WO 01/46151, WO 02/074726 and WO 02/098878). Such additional compounds also include cholesterol-lowering drugs such as the statins, e.g. simvastatin. Such additional compounds similarly include compounds known to modify the production or processing of Aβ in the brain (“amyloid modifiers”), such as compounds which modulate the secretion of Aβ (including γ-secretase inhibitors, γ-secretase modulators and β-secretase inhibitors), compounds which inhibit the aggregation of Aβ, and antibodies which selectively bind to Aβ. Such additional compounds further include growth hormone secretagogues, e.g. as described in WO 2004/080459.

In this embodiment of the invention, the amyloid modifier may be a compound which inhibits the secretion of Aβ, for example an inhibitor of γ-secretase (such as those disclosed in WO 01/90084, WO 02/30912, WO 01/70677, WO 03/013506, WO 02/36555, WO 03/093252, WO 03/093264, WO 03/093251, WO 03/093253, WO 2004/039800, WO 2004/039370, WO 2005/030731, WO 2005/014553, WO 2004/089911, WO 02/081435, WO 02/081433, WO 03/018543, WO 2004/031137, WO 2004/031139, WO 2004/031138, WO 2004/101538, WO 2004/101539 and WO 02/47671), or a β-secretase inhibitor (such as those disclosed in WO 03/037325, WO 03/030886, WO 03/006013, WO 03/006021, WO 03/006423, WO 03/006453, WO 02/002122, WO 01/70672, WO 02/02505, WO 02/02506, WO 02/02512, WO 02/02520, WO 02/098849 and WO 02/100820), or any other compound which inhibits the formation or release of Aβ including those disclosed in WO 98/28268, WO 02/47671, WO 99/67221, WO 01/34639, WO 01/34571, WO 00/07995, WO 00/38618, WO 01/92235, WO 01/77086, WO 01/74784, WO 01/74796, WO 01/74783, WO 01/60826, WO 01/19797, WO 01/27108, WO 01/27091, WO 00/50391, WO 02/057252, US 2002/0025955 and US2002/0022621, and also including GSK-3 inhibitors, particularly GSK-3α inhibitors, such as lithium, as disclosed in Phiel et al, Nature, 423 (2003), 435-9.

Alternatively, the amyloid modifier may be a compound which modulates the action of γ-secretase so as to selectively attenuate the production of Aβ(1-42). Compounds reported to show this effect include certain non-steroidal antiinflammatory drugs (NSAIDs) and their analogues (see WO 01/78721 and US 2002/0128319 and Weggen et al Nature, 414 (2001) 212-16; Morihara et al, J. Neurochem., 83 (2002), 1009-12; and Takahashi et al, J. Biol. Chem., 278 (2003), 18644-70), and compounds which modulate the activity of PPARα and/or PPARδ (WO 02/100836). Further examples of γ-secretase modulators are disclosed in WO 2005/054193, WO 2005/013985, WO 2005/108362, WO 2006/008558 and WO 2006/043064.

Alternatively, the amyloid modifier may be a compound which inhibits the aggregation of Aβ or otherwise attenuates is neurotoxicicity. Suitable examples include chelating agents such as clioquinol (Gouras and Beal, Neuron, 30 (2001), 641-2) and the compounds disclosed in WO 99/16741, in particular that known as DP-109 (Kalendarev et al, J. Pharm. Biomed. Anal., 24 (2001), 967-75). Other inhibitors of Aβ aggregation suitable for use in the invention include the compounds disclosed in WO 96/28471, WO 98/08868 and WO 00/052048, including the compound known as Apan™ (Praecis); WO 00/064420, WO 03/017994, WO 99/59571 (in particular 3-aminopropane-1-sulfonic acid, also known as tramiprosate or Alzhemed™); WO 00/149281 and the compositions known as PTI-777 and PTI-00703 (ProteoTech); WO 96/39834, WO 01/83425, WO 01/55093, WO 00/76988, WO 00/76987, WO 00/76969, WO 00/76489, WO 97/26919, WO 97/16194, and WO 97/16191. Further examples include phytic acid derivatives as disclosed in U.S. Pat. No. 4,847,082 and inositol derivatives as taught in US 2004/0204387.

Alternatively, the amyloid modifier may be an antibody which binds selectively to Aβ. Said antibody may be polyclonal or monoclonal, but is preferably monoclonal, and is preferably human or humanized. Preferably, the antibody is capable of sequestering soluble Aβ from biological fluids, as described in WO 03/016466, WO 03/016467, WO 03/015691 and WO 01/62801. Suitable antibodies include humanized antibody 266 (described in WO 01/62801) and the modified version thereof described in WO 03/016466. Suitable antibodies also include those specific to Aβ-derived diffusible ligands (ADDLS), as disclosed in WO 2004/031400.

As used herein, the expression “in combination with” requires that therapeutically effective amounts of both the BRSK inhibitor and the additional compound are administered to the subject, but places no restriction on the manner in which this is achieved. Thus, the two species may be combined in a single dosage form for simultaneous administration to the subject, or may be provided in separate dosage forms for simultaneous or sequential administration to the subject. Sequential administration may be close in time or remote in time, e.g. one species administered in the morning and the other in the evening. The separate species may be administered at the same frequency or at different frequencies, e.g. one species once a day and the other two or more times a day. The separate species may be administered by the same route or by different routes, e.g. one species orally and the other parenterally, although oral administration of both species is preferred, where possible. When the additional compound is an antibody, it will typically be administered parenterally and separately from the BRSK inhibitor.

### EXAMPLES

### IMAP Screening Method for BRSK Inhibitors

MATERIALS

| | |
|---|---|
| ATP | Sigma # A6419 |
| FAM-Cdc25C peptide | Molecular Devices # R7275 |
| IMAP Screening Express Kit with | Molecular Devices #R8127 |
| Progressive Binding System | |
| 384-well black plates | Corning #3710 |
| Staurosporine | Upstate # 19-123 |
| BRSK1 (human, recombinant) | Upstate # 14-675 |
| BRSK2 (human, recombinant) | University of Dundee |
| DTT | Sigma # D9779 |

### Method

1. Defrost ATP, DTT and FAM-Cdc25C substrate

2. Defrost enzymes on ice

3. Prepare complete reaction buffer with 1 mM DTT
(i) 99 ml 1×IMAP reaction buffer
(ii) 1 ml 100 mM DTT

4. Prepare inhibitors
(i) dilute compound stocks to 1 mM in DMSO
(ii) serially dilute compounds in DMSO to give concentrations ranging from 40× final concentration range
(iii) dilute 1 in 10 using complete reaction buffer to give 4× compound concentration range
(iv) dilute 1 in 4 into assay plates to give final concentration range

5. Prepare enzyme
(i) dilute enzyme to 4× final (120 nM) in complete reaction buffer
(ii) transfer 5 μl to assay plate
(iii) shake for 2 min at RT and leave to incubate at RT for 5 min

6. Prepare substrate/ATP mix
(i) add ATP to complete reaction buffer to give 200 μM (2× final)
(ii) add Cdc25C substrate to give 100 nM (2× final)
(iii) transfer 10 μl to assay plates
(iv) Shake for 2 min at RT, protected from light

7. Incubate for 1 h at RT, protected from light

8. Prepare IMAP beads
(i) for 100 ml add 75 ml 1× buffer A and 25 ml 1× buffer B
(ii) add 167 μl beads ( 1/600 dilution)
(iii) add 60 μl to assay wells

9. Incubate for 1 h at RT, protected from light

10. Read on Fluorescence Polarization-enabled reader

### HTRF Screening Method for BRSK Inhibitors

MATERIALS

| | |
|---|---|
| ATP | Sigma |
| Cdc25C biotinylated peptide | Cell Signalling technologies |
| Phospho-Ser 14-3-3 Binding Motif mouse ab | Cell Signalling technologies |
| Europium cryptate ab conjugation (EuK) | CisBio |
| Streptavidin XL^{ent!} 665 | Cisbio |
| 384-well low volume black plates | Greiner Bio-One |
| Staurosporine | Upstate |
| BRSK1 (human, recombinant) | Upstate |
| BRSK2 (human, recombinant) | University of Dundee |
| DTT | Sigma |

### Method

1. Defrost ATP, DTT, and biotin-cdc25C substrate

2. Defrost enzymes on ice

3. Prepare kinase reaction buffer
(i) 25 mM Tris-HCL, pH7.5
(ii) 5 mM β-glycerophosphate
(iii) 2 mM DTT
(iv) 0.1 mM Na₃VO₄
(v) 5 mM MgCl₂
(vi) supplement with 0.1% (w/v) BSA and 0.01% (v/v) Tween-20 on day of assay

4. Prepare inhibitors
(vii) dilute compound stocks to 1 mM in DMSO
(viii) serially dilute compounds in DMSO to give 30× final concentration range
(ix) dilute 1 in 10 in kinase reaction buffer to give 3× final concentration range
(x) dilute 1 in 3 into assay plates to give final concentration range

5. Prepare enzyme
(xi) dilute enzyme to 30 nM (3× final) in kinase reaction buffer
(xii) transfer 5 μl to assay plate
(xiii) shake for 2 min at RT and leave to incubate at RT for 5 min

6. Prepare substrate/ATP mix
(xiv) add 3×ATP to reaction buffer
(xv) add biotin-Cdc25C substrate to give 3× final concentration
(xvi) transfer 5 μl to assay plates
(xvii) shake for 2 min at RT

7. Incubate for 2 h at 30° C.

8. Prepare stop buffer
(xviii) 50 mM Tris-HCl, pH8
(xix) 0.1% (w/v) BSA
(xx) 0.5M KF
(xxi) 0.01% (v/v) Tween-20
(xxii) 15 mM EDTA
(xxiii) 1:215 EuK labelled ab
(xxiv) 1:500 1 mg/ml SA-XL^{ent} 665

9. Transfer 5 μl stop buffer to assay plates

10. Incubate overnight at 4° C.

11. Read plates on HTRF enabled reader

### SYNTHESIS EXAMPLES

### Example 1

### 3-{5-[(4-acetylpiperazin-1-yl)methyl]-1H-indol-2-yl}-1H-indazole-6-carbonitrile

### Step 1: 3-amino-4-methylbenzonitrile (1-2)

A mixture of 3-nitro-p-tolunitrile (9.30 g, 57.4 mmol, 1 equiv) and 10% Pd/C (4.00 g, 3.76 mmol, 0.066 equiv) in ethanol (100 mL) was stirred under a hydrogen balloon at 23° C. for 20 h. The catalyst was filtered onto a pad a celite and washed with EtOAc (300 mL). The filtrate was concentrated to give 3-amino-4-methylbenzonitrile (1-2) as an off-white solid. ¹H NMR (300 MHz, CDCl₃) δ 7.10 (d, 1H, J=7.6 Hz), 6.98 (dd, 1H, J=7.9, 1.5 Hz), 6.89 (d, 1H, J=1.5 Hz), 3.78 (br s, 2H), 2.20 (s, 3H). LRMS m/z (M+H+CH₃CN) 174.2 found, 174.1 required.

### Step 2: 1H-indazole-6-carbonitrile

A solution of sodium nitrite (4.14 g, 59.9 mmol, 1.10 equiv) in water (20 mL) was added slowly to a pre-cooled (−10° C.) mixture of 3-amino-4-methylbenzonitrile (1-2, 7.2 g, 54.5 mmol, 1 equiv) and concentrated aqueous hydrochloric acid solution (12 M, 13.6 mL, 163 mmol, 3.00 equiv) in water (50 mL) at a rate that kept the reaction mixture temperature below 0° C. Following the addition, the reaction mixture was stirred at −5° C. for 30 min, then filtered. A solution of sodium tetrafluoroborate (17.9 g, 163 mmol, 3.00 equiv) in water (100 mL) was immediately added to the cold filtrate. The precipitate was filtered and washed with ice-cold water (30 mL). The remaining solid was air-dried to give 5-cyano-2-methylbenzenediazonium tetrafluoroborate as a white solid. A suspension of this product (11.7 g, 50.7 mmol, 1 equiv), potassium acetate (12.4 g, 127 mmol, 2.50 equiv) and 18-crown-6 (1.34 g, 5.07 mmol, 0.100 equiv) in chloroform (150 mL) was stirred at 23° C. for 20 h. The reaction mixture was filtered and concentrated. The residue was partitioned between water and EtOAc (300 mL). The organic layer was washed with brine, dried over sodium sulfate and concentrated to give 1H-indazole-6-carbonitrile (1-3) as a light yellow solid. ¹H NMR (300 MHz, CDCl₃) δ 10.37 (br s, 1H), 8.19 (d, 1H, J=0.9 Hz), 7.90 (d, 1H, J=1.5 Hz), 7.88 (dd, 1H, J=8.5, 0.9 Hz), 7.42 (dd, 1H, J=8.3, 1.5 Hz). LRMS m/z (M+H+CH₃CN) 185.1 found, 185.0 required.

### Step 3: 3-iodo-1H-indazole-6-carbonitrile (1-4)

A mixture of 1H-indazole-6-carbonitrile (1-3, 4.7 g, 32.8 mmol, 1 equiv), iodine (18.3 g, 72.2 mmol, 2.20 equiv) and potassium hydroxide (4.42 g, 78.8 mmol, 2.40 equiv) in DMF (75 mL) was stirred at 23° C. for 5 h. The reaction mixture was partitioned between a 1:1 aqueous mixture of saturated sodium chloride solution and saturated sodium thiosulfate solution and ethyl acetate (2×300 mL). The combined organic layers were washed with water then brine, dried over sodium sulfate and concentrated to give 3-iodo-1H-indazole-6-carbonitrile (1-4) as a light yellow solid. ¹H NMR (300 MHz, CDCl₃) δ 13.29 (br s, 1H), 7.90 (s, 1H), 7.59 (d, 1H, J=8.2 Hz), 7.38 (dd, 1H, J=8.5, 0.9 Hz). LRMS m/z (M+H+CH₃CN) 311.1 found, 311.0 required.

### Step 4: tert-butyl 5-({[tert-butyl(dimethyl)silyl]oxy}methyl)-1H-indole-1-carboxylate (1-6)

A solution of lithium aluminum hydride in THF (1.0 M, 180 mL, 180 mmol, 1.50 equiv) was added over 20 min to a solution of methyl 1H-indole-5-carboxylate (1-5, 21.0 g, 120 mmol, 1 equiv) in THF (400 mL) at 0° C. The reaction mixture was allowed to warm to 23° C. then heated at 40° C. for 2 h. The reaction mixture was poured into ice water (1 liter) then extracted with ethyl acetate (2×500 mL). The combined organic layers were washed with brine, dried over sodium sulfate and concentrated to provide 1H-indol-5-ylmethanol as a white solid. A solution of 1H-indol-5-ylmethanol (18.0 g, 122 mmol, 1 equiv), tert-butyldimethylsilyl chloride (20.3 g, 135 mmol, 1.10 equiv), triethylamine (43.4 mL, 245 mmol, 2.00 equiv) and 4-(dimethylamino)pyridine (1.49 g, 12.2 mmol, 0.100 equiv) in dichloromethane (300 mL) was stirred at 23° C. for 2 h. The reaction mixture was concentrated and the residue partitioned between saturated aqueous sodium bicarbonate solution and ethyl acetate (400 mL). The organic layer was washed with aqueous 0.5 N hydrochloride acid solution then brine, dried over sodium sulfate and concentrated. A solution of the residual oil (5-({[tert-butyl(dimethyl)silyl]oxy}methyl)-1H-indole), di-tert-butyl dicarbonate (29.4 g, 135 mmol, 1.10 equiv) and 4-(dimethylamino)pyridine (1.49 g, 12.2 mmol, 0.100 equiv) in dichloromethane (300 mL) was stirred at 23° C. for 3 h. The reaction mixture was concentrated and the residue purified by flash column chromatography (hexanes initially, grading to 20% ethyl acetate in hexanes) to provide tert-butyl 5-({[tert-butyl(dimethyl)silyl]oxy}methyl)-1H-indole-1-carboxylate (1-6) as a colorless oil. ¹H NMR (500 MHz, CDCl₃) δ 8.07 (br d, 1H, J=6.8 Hz), 7.58 (br d, 1H, J=3.2 Hz), 7.52 (s, 1H), 7.25 (CHCl₃ obscured dd, 1H), 6.54 (d, 1H, J=3.7 Hz), 4.82 (s, 2H), 1.67 (s, 9H), 0.95 (s, 9H), 0.11 (s, 6H).

### Step 5: 1-(tert-butoxycarbonyl)-5-({[tert-butyl(dimethyl)silyl]oxy}methyl)-1H-indol-2-ylboronic Acid (1-7)

A solution of LDA in THF (0.773 M, 200 mL, 155 mmol, 1.30 equiv) at −78° C. was added via cannula to a solution of tert-butyl 5-({[tert-butyl(dimethyl)silyl]oxy}methyl)-1H-indole-1-carboxylate (1-6, 43.0 g, 119 mmol, 1 equiv) in THF (400 mL) at −78° C., and the resulting mixture was stirred for 45 min. Trimethylborate (27.0 mL, 238 mmol, 2.00 equiv) was added and the resulting mixture was warmed to 0° C. and held at that temperature for 30 min. The reaction mixture was partitioned between saturated aqueous ammonium chloride solution and ethyl acetate (2×200 mL). The combined organic layers were washed with brine, dried over sodium sulfate and concentrated to provide 1-(tert-butoxycarbonyl)-5-({[tert-butyl(dimethyl)silyl]oxy}methyl)-1H-indol-2-ylboronic acid (1-7) as an off-white solid. ¹H NMR (500 MHz, CD₃OD) δ 8.05 (d, 1H, J=8.6 Hz), 7.51 (s, 1H), 7.26 (dd, 1H, J=8.6, 1.7 Hz), 6.62 (s, 1H), 4.82 (s, 2H), 1.68 (s, 9H), 0.95 (s, 9H), 0.11 (s, 6H).

### Step 6: tert-butyl 2-(6-cyano-1H-indazol-3-yl)-5-(hydroxymethyl)-1H-indole-1-carboxylate (1-8)

A deoxygenated mixture of 3-iodo-1H-indazole-6-carbonitrile (1-4, 5.00 g, 18.6 mol, 1 equiv), 1-(tert-butoxycarbonyl)-5-({[tert-butyl(dimethyl)silyl]oxy}methyl)-1H-indol-2-ylboronic acid (1-7, 9.04 g, 22.3 mmol, 1.20 equiv), lithium chloride (2.36 g, 55.8 mmol, 3.00 equiv), aqueous sodium carbonate solution (2 M, 46.5 mL, 92.9 mmol, 5.00 equiv), and Pd(PPh₃)₄ (1.07 g, 0.929 mol, 0.050 equiv) in dioxane (300 mL) was heated under nitrogen at 90° C. for 20 h. Additional 1-(tert-butoxycarbonyl)-5-({[tert-butyl(dimethyl)silyl]oxy}methyl)-1H-indol-2-ylboronic acid (1-7, 3.77 g, 9.30 mmol, 0.500 equiv) was added and heating was continued for 5 h. The reaction mixture was partitioned between half-saturated aqueous sodium chloride solution and ethyl acetate (2×300 mL). The combined organic layers were washed with brine, dried over sodium sulfate and concentrated. A solution of the residue and triethylamine trihydrofluoride (15.4 mL, 92.9 mmol, 5.00 equiv) in acetonitrile (300 mL) was heated at 50° C. for 3 h. The reaction mixture was concentrated and the residue partitioned between saturated aqueous sodium bicarbonate solution and ethyl acetate (2×300 mL). The combined organic layers were washed with brine, dried over sodium sulfate and concentrated. The residue was purified by flash column chromatography (hexanes initially, grading to 60% EtOAc in hexanes) to provide tert-butyl 2-(6-cyano-1H-indazol-3-yl)-5-(hydroxymethyl)-1H-indole-1-carboxylate (1-8) as an orange foam. ¹H NMR (300 MHz, CDCl₃) δ 12.03 (br s, 1H), 8.27 (d, 1H, J=8.5 Hz), 7.76 (d, 1H, J=8.5 Hz), 7.69 (br s, 1H), 7.59 (br s, 1H), 7.48 (dd, 1H, J=8.5, 1.7 Hz), 7.39 (dd, 1H, J=8.5, 1.0 Hz), 6.91 (s, 1H), 4.85 (s, 2H), 1.20 (s, 9H). LRMS m/z (M+H−t-Bu) 333.3 found, 333.1 required.

### Step 7: tert-butyl 2-(6-cyano-1H-indazol-3-yl)-5-formyl-1H-indole-1-carboxylate (1-9)

A mixture of tert-butyl 2-(6-cyano-1H-indazol-3-yl)-5-(hydroxymethyl)-1H-indole-1-carboxylate (1-8, 4.00 g, 10.3 mmol, 1 equiv) and manganese(IV) oxide (4.48 g, 51.5 mmol, 5.00 equiv) in dichloromethane (300 mL) was heated at 40° C. for 2 h. Additional MnO₂ (4.48 g, 51.5 mmol, 5.00 equiv) was added and heating was continued for 2 h. The solids were filtered and washed repeatedly with dichloromethane (400 mL total) and ethyl acetate (400 mL total). The combined filtrate was concentrated and the residue purified by flash column chromatography (hexanes initially, grading to 40% EtOAc in hexanes) to provide tert-butyl 2-(6-cyano-1H-indazol-3-yl)-5-formyl-1H-indole-1-carboxylate (1-9) as an off-white solid. ¹H NMR (300 MHz, CDCl₃) δ 10.49 (br s, 1H), 10.12 (s, 1H), 8.42 (d, 1H, J=8.8 Hz), 8.18 (d, 1H, J=1.2 Hz), 7.96 (dd, 1H, J=8.8, 1.8 Hz), 7.94 (br s, 1H), 7.78 (d, 1H, J=8.6 Hz), 7.46 (dd, 1H, J=8.5, 1.2 Hz), 6.91 (s, 1H), 1.21 (s, 9H). LRMS m/z (M+H−t-Bu) 331.2 found, 331.1 required.

### Step 8: 3-{5-[(4-acetylpiperazin-1-yl)methyl]-1H-indol-2-yl}-1H-indazole-6-carbonitrile

A mixture of tert-butyl 2-(6-cyano-1H-indazol-3-yl)-5-formyl-1H-indole-1-carboxylate (1-9, 99 mg, 0.26 mmol, 1 equiv), 1-acetylpiperazine (49 mg, 0.38 mmol, 1.5 equiv), and sodium triacetoxyborohydride (81 mg, 0.38 mmol, 1.5 equiv) in 1,2-dichloromethane (5 mL) was stirred at 23° C. for 40 min. Additional 1-acetylpiperazine (49 mg, 0.38 mmol, 1.50 equiv), and sodium triacetoxyborohydride (81 mg, 0.38 mmol, 1.5 equiv) were added and stirring was continued for 2 h. The reaction was quenched with a dilute aqueous sodium bicarbonate solution and partitioned between water and ethyl acetate (3×50 mL). The combined organic layers were dried over sodium sulfate and concentrated. The residual oil was dissolved in a 1:1 mixture of dichloromethane and trifluoroacetic acid and allowed to stand for 3 h. The solution was concentrated and the residue was purified by reverse phase liquid chromatography (H₂O/CH₃CN gradient w/0.1% TFA present). The desired fractions were partitioned between saturated aqueous sodium bicarbonate solution and ethyl acetate (2×50 mL). The combined organic layers were washed with brine then dried over sodium sulfate and concentrated to give 3-{5-[(4-acetylpiperazin-1-yl)methyl]-1H-indol-2-yl}-1H-indazole-6-carbonitrile as a free base (light brown solid). ¹H NMR (300 MHz, CDCl₃) δ 10.75 (br s, 1H), 9.08 (br s, 1H), 8.22 (d, 1H, J=8.5 Hz), 7.89 (br s, 1H), 7.60 (br s, 1H), 7.51 (dd, 1H, J=8.5, 1.2 Hz), 7.40 (d, 1H, J=8.5 Hz), 7.23 (dd, 1H, J=8.5, 1.7 Hz), 7.10 (d, 1H, J=1.7 Hz), 3.65 (s, 2H), 3.49 (m, 2H), 2.50 (m, 4H), 2.11 (s, 3H). LRMS m/z (M+H) 399.4 found, 399.2 required.

### Examples 2-9

The following were prepared by simple modification of the method of Example 1:

### Example 2

### 3-[5-(piperazin-1-ylmethyl)-1H-indol-2-yl]-1H-indazole-6-carbonitrile

LRMS m/z (M+H) 357.4 found, 357.2 required

### Example 3

### 3-(5-{[(2-aminoethyl)amino]methyl}-1H-indol-2-yl)-1H-indazole-6-carbonitrile

LRMS m/z (M+H) 331.3 found, 331.2 required

### Example 4

### 3-(5-{[(4-aminocyclohexyl)amino]methyl}-1H-indol-2-yl)-1H-indazole-6-carbonitrile

LRMS m/z (M+H) 385.3 found, 385.2 required

### Example 5

### 3-(4-{[4-(amino methyl)piperidin-1-yl]methyl}-1H-indol-2-yl)-1H-indazole-6-carbonitrile

LRMS m/z (M+H) 385.5 found, 385.2 required

### Example 6

### 3-[4-({[1-(pyridin-4-ylmethyl)piperidin-4-yl]amino}methyl)-1H-indol-2-yl]-1H-indazole-6-carbonitrile

LRMS m/z (M+H) 462.2 found, 462.2 required.

### Example 7

### 3-[4-({[(1-methylpiperidin-4-yl)methyl]amino}methyl)-1H-indol-2-yl]-1H-indazole-6-carbonitrile

LRMS m/z (M+H) 399.2 found, 399.2 required

### Example 8

### 3-[5-(morpholin-4-ylmethyl)-1H-indol-2-yl]-1H-indazole-6-carbonitrile

LRMS m/z (M+H) 358.4 found, 358.2 required

### Example 9

### 3-[4-({[2-(tert-butylthio)ethyl]amino}methyl)-1H-indol-2-yl]-1H-indazole-6-carbonitrile

LRMS m/z (M+H) 404.2 found, 404.2 required

### Example 10

### Methyl 3-(4-{[2-(6-chloro-1H-indazol-3-yl)-1H-indol-4-yl]methyl}piperazin-1-yl)butanoate

prepared as described in WO 2003/024969.

### Example 11

### 3-{5-[(4-acetylpiperazin-1-yl)methyl]-1H-indol-2-yl}-6-(1H-tetrazol-5-yl)-1H-indazole

A mixture of 3-{5-[(4-acetylpiperazin-1-yl)methyl]-1H-indol-2-yl}-1H-indazole-6-carbonitrile (Example 1, 90 mg, 0.23 mmol, 1 equiv), and azidotrimethyltin (479 mg, 2.33 mmol, 10.0 equiv) in a 1:5 mixture of dimethyl acetamide and toluene was heated at 110° C. for 18 h. The reaction mixture was concentrated and the residue was purified by reverse-phase LC(H₂O/CH₃CN gradient w/0.1% TFA present)3-{5-[(4-acetylpiperazin-1-yl)methyl]-1H-indol-2-yl}-6-(1H-tetraazol-5-yl)-1H-indazole (8-1) as a TFA salt (light yellow solid). LRMS m/z (M+H) 442.6 found, 442.2 required.

### Example 12

### 3-(5-{[4-(methyl sulfonyl)piperazin-1-yl]methyl}-1H-indol-2-yl)-6-(1H-tetraazol-5-yl)-1H-indazole

prepared in analogous manner to Example 11.

LRMS m/z (M+H) 478.4 found, 478.2 required.

### Example 13

### Methyl 3-[5-(morpholin-4-ylmethyl)-1H-indol-2-yl]-1H-indazole-6-carboxylate

### Step 1: methyl 1H-indazole-6-carboxylate

A solution of sodium nitrite (4.18 g, 60.5 mmol, 2.00 equiv) in water (25 mL) was added slowly to a pre-cooled (−10° C.) mixture of methyl 3-amino-4-methylbenzoate (5.00 g, 30.3 mmol, 1.00 equiv) and concentrated aqueous hydrochloric acid solution (12 M, 7.6 mL, 90.8 mmol, 3.00 equiv) in water (50 mL) at a rate that kept the reaction mixture temperature below 0° C. Following the addition, the reaction mixture was stirred at −5° C. for 30 min, then filtered. A solution of sodium tetrafluoroborate (9.97 g, 90.8 mmol, 3.00 equiv) in water (40 mL) was immediately added to the cold filtrate. The precipitate was filtered and washed with ice-cold water (50 mL). The remaining solid was air-dried to give 5-(methoxycarbonyl)-2-methylbenzenediazonium tetrafluoroborate as a white solid. A suspension of this product (5.00 g, 28.2 mmol, 1 equiv), potassium acetate (6.92 g, 70.6 mmol, 2.50 equiv) and 18-crown-6(746 mg, 2.82 mmol, 0.100 equiv) in chloroform (75 mL) was stirred at 23° C. for 72 h. The reaction mixture was filtered and concentrated. The residue was partitioned between water and EtOAc (300 mL). The organic layer was washed with brine, dried over sodium sulfate and concentrated to give methyl 1H-indazole-6-carboxylate (2-2) as an orange solid. ¹H NMR (300 MHz, CDCl₃) [symbol] 8.20 (s, 1H), 8.16 (d, 1H, J=0.9 Hz), 7.89 (d, 1H, 8.5 Hz), 7.68, (dd, 1H, J=8.5, 1.5 Hz), 3.90 (s, 3H). LRMS m/z (M+H) 177.1 found, 177.1 required.

### Step 2: methyl 3-iodo-1H-indazole-6-carboxylate (2-3)

A mixture of methyl 1H-indazole-6-carboxylate (2-2, 3.99 g, 22.6 mmol, 1 equiv), iodine (12.6 g, 49.8 mmol, 2.20 equiv) and potassium hydroxide (3.05 g, 54.4 mmol, 2.40 equiv) in DMF (70 mL) was stirred at 23° C. for 15 h. The reaction mixture was partitioned between a 1:1 aqueous mixture of saturated sodium chloride solution and saturated sodium thiosulfate solution and ethyl acetate (2×300 mL). The combined organic layers were washed with water then brine, dried over sodium sulfate and concentrated to give methyl 3-iodo-1H-indazole-6-carboxylate (2-3) as a yellow solid. ¹H NMR (300 MHz, CDCl₃) δ 11.6 (br s, 1H) 8.27 (d, 1H, J=2.1 Hz), 7.90 (dd, 1H, J=8.6, 1.2 Hz), 7.57 (d, 1H, J=8.6 Hz), 3.98 (s, 3H).

LRMS m/z (M+H) 303.1 found, 303.0 required.

### Step 3: methyl 3-[1-(tert-butoxycarbonyl)-5-(hydroxymethyl)-1H-indol-2-yl]-2,3-dihydro-1H-indazole-6-carboxylate (2-4)

A deoxygenated mixture of methyl 3-iodo-1H-indazole-6-carboxylate (2-3, 2.11 g, 6.97 mmol, 1 equiv), 1-(tert-butoxycarbonyl)-5-({[tert-butyl(dimethyl)silyl]oxy}methyl)-1H-indol-2-ylboronic acid (1-7, 3.39 g, 8.37 mmol, 1.20 equiv), lithium chloride (887 mg, 20.9 mmol, 3.00 equiv), aqueous sodium carbonate solution (2M, 17.4 mL, 34.9 mmol, 5.00 equiv), and Pd(PPh₃)₄ (403 mg, 0.349 mmol, 0.050 equiv) in dioxane (20 mL) was heated under nitrogen at 90° C. for 20 h. The reaction mixture was partitioned between half-saturated aqueous sodium chloride solution and ethyl acetate (2×200 mL). The combined organic layers were washed with brine, dried over sodium sulfate and concentrated. A solution of the residue and triethylamine trihydrofluoride (5.68 mL, 34.9 mmol, 5.00 equiv) in acetonitrile (50 mL) was heated at 50° C. for 6 h. The reaction mixture was concentrated and the residue partitioned between saturated aqueous sodium bicarbonate solution and ethyl acetate (2×200 mL). The combined organic layers were washed with brine, dried over sodium sulfate and concentrated. The residue was purified by flash column chromatography (hexanes initially, grading to 60% EtOAc in hexanes) to provide methyl 3-[1-(tert-butoxycarbonyl)-5-(hydroxymethyl)-1H-indol-2-yl]-2,3-dihydro-1H-indazole-6-carboxylate (2-4) as a dark-colored solid. LRMS m/z (M+H−t-Bu) 366.3 found, 366.2 required.

### Step 4: methyl 3-[1-(tert-butoxycarbonyl)-5-formyl-1H-indol-2-yl]-1H-indazole-6-carboxylate (2-5)

A mixture of methyl 3-[1-(tert-butoxycarbonyl)-5-(hydroxymethyl)-1H-indol-2-yl]-2,3-dihydro-1H-indazole-6-carboxylate (2-4, 1.69 g, 4.01 mmol, 1 equiv) and manganese(IV) oxide (1.74 g, 20.0 mmol, 5.00 equiv) in dichloromethane (50 mL) was heated at 40° C. for 2 h. Additional MnO₂ (1.05 g, 12.0 mmol, 3.00 equiv) was added and heating was continued for 2 h. The solids were filtered and washed repeatedly with dichloromethane (300 mL) and ethyl acetate (300 mL). The combined filtrate was partitioned between aqueous half-saturated sodium chloride solution and ethyl acetate (2×200 mL). The combined organic layers were dried over sodium sulfate and concentrated to provide methyl 3-[1-(tert-butoxycarbonyl)-5-formyl-1H-indol-2-yl]-1H-indazole-6-carboxylate (2-5) as a dark-colored solid. LRMS m/z (M+H−t-Bu) 364.3 found, 364.2 required.

### Step 5 methyl 3-[5-(morpholin-4-ylmethyl)-1H-indol-2-yl]-1H-indazole-6-carboxylate

A mixture of methyl 3-[1-(tert-butoxycarbonyl)-5-formyl-1H-indol-2-yl]-1H-indazole-6-carboxylate (2-5, 950 mg, 2.23 mmol, 1 equiv), morpholine (0.790 mL, 9.06 mmol, 4.00 equiv), and sodium triacetoxyborohydride (1.92 g, 9.06 mmol, 4.00 equiv) in 1,2-dichloroethane (60 mL) was stirred at 23° C. under nitrogen for 14 h. The reaction was partitioned between saturated aqueous sodium bicarbonate solution and ethyl acetate (3×100 mL). The combined organic layers were dried over sodium sulfate and concentrated. The residue was dissolved in a 1:1 mixture of dichloromethane and trifluoroacetic acid and allowed to stand for 3 h. The solution was concentrated and the residue was purified by reverse phase liquid chromatography (H₂O/CH₃CN gradient w/0.1% TFA present) to give methyl 3-[5-(morpholin-4-ylmethyl)-1H-indol-2-yl]-1H-indazole-6-carboxylate as a TFA salt (light brown solid). ¹H NMR (300 MHz, DMSO-d₆) δ 9.61 (br s, 1H), 8.34 (d, 1H, J=8.3 Hz), 8.23 (s, 1H), 7.83 (dd, 1H, J=9.7, 1.2 Hz), 7.75 (s, 1H), 7.54 (dd, 1H, J=8.1, 1.2 Hz), 7.27 (d, 1H, J=1.2 Hz), 7.25 (dd, 1H, J=7.1. 1.2 Hz), 4.43 (m, 2H), 3.97 (m, 2H), 3.93 (s, 3H), 3.63 (m, 2H), 3.31 (s obscured by H₂O peak, 2H), 3.16 (m, 2H).

LRMS m/z (M+H) 391.5 found, 391.2 required.

### Example 14

### N-methyl-3-[5-(morpholin-4-ylmethyl)-1H-indol-2-yl]-1H-indazole-6-carboxamide

### Step 1: 1H-indazole-6-carboxylic Acid

A mixture of 1H-indazole-6-carbonitrile (1-3, 1.50 g, 10.5 mmol, 1.00 equiv), and sodium hydroxide (1.26 g, 31.4 mmol, 3 equiv) in a 1:1 mixture of ethanol and 1N aqueous sodium hydroxide (5 mL) was heated to 80° C. for 4 h. The reaction mixture was partitioned between saturated aqueous sodium bicarbonate solution and ethyl acetate (4×50 mL). The organic layer was discarded and the aqueous layer was acidified to pH 5 and washed with ethyl acetate (2×50 mL). The combined organic layers were dried over sodium sulfate and concentrated to give 1H-indazole-6-carboxylic acid (4-1) as an off-white solid. The LRMS m/z (M+H) 163.1 found, 163.0 required.

### Step 2: N-methyl-1H-indazole-6-carboxamide

A mixture of 1H-indazole-6-carboxylic acid (4-1, 1.11 g, 6.85 mmol, 1 equiv), N,N-diisopropylethylamine (2.65 g, 20.5 mmol, 3.00 equiv), (1H-1,2,3-benzotriazol-1-yloxy)(tripyrrolidin-1-yl)phosphonium hexafluorophosphate (Pybop, 5.34 g, 10.3 mmol, 1.50 equiv) and methylamine (10.3 mL (2M in THF), 20.5 mmol, 3.00 equiv) in DMF (10 mL) was stirred at 23° C. for 14 h. The reaction mixture was partitioned between a 1:1 aqueous mixture of sodium chloride solution and sodium bicarbonate solution and ethyl acetate (2×50 mL). The aqueous layer was then acidified to pH 5 and washed with ethyl acetate (2×50 mL). The combined organic layers were dried over sodium sulfate and concentrated. The residue was purified by flash column chromatography (hexanes initially, grading to 100% EtOAc, then 10% MeOH in EtOAc) to give N-methyl-1H-indazole-6-carboxamide (4-2) as a light brown solid. LRMS m/z (M+H) 176.2 found, 176.1 required.

### Step 3: 3-iodo-N-methyl-1H-indazole-6

A mixture of N-methyl-1H-indazole-6-carboxamide (4-2, 3.30 g, 18.8 mmol, 1 equiv), iodine (10.5 g, 41.4 mmol, 2.20 equiv) and potassium hydroxide (2.54 g, 45.2 mmol, 2.40 equiv) in DMF (10 mL) was stirred at 23° C. for 15 h. The reaction mixture was partitioned between a 1:1 aqueous mixture of saturated sodium chloride solution and saturated sodium thiosulfate solution and ethyl acetate (2×300 mL). The combined organic layers were washed with water then brine, dried over sodium sulfate and concentrated. The residue was triturated with acetonitrile to give 3-iodo-N-methyl-1H-indazole-6-carboxamide (4-3) as a light brown solid. LRMS m/z (M+H) 302.2 found, 302.0 required.

### Step 4: N-methyl-3-[5-(morpholin-4-ylmethyl)-1H-indol-2-yl]-1H-indazole-6-carboxamide

The product of step 3 was coupled with 1-(tert-butoxycarbonyl)-5-({[tert-butyl(dimethyl)silyl]oxy}methyl)-1H-indol-2-ylboronic acid, oxidised to the aldehyde, and reacted with morpholine and sodium triacetoxyborohydride by the procedures described in Example 1 Steps 6-8.

LRMS m/z (M+H) 390.4 found, 390.2 required

### Example 15

### 6-(4,5-dihydro-1,3-oxazol-2-yl)-3-[5-(morpholin-4-ylmethyl)-1H-indol-2-yl]-1H-indazole

A mixture of 1H-indazole-6-carbonitrile (1-3, 100 mg, 0.70 mmol, 1 equiv), ethanolamine (0.21 mL, 3.5 mmol, 5.00 equiv) and cadmium acetate dehydrate (5 mg, 0.02 mmol, 0.03 equiv) was heated neat at 140° C. for 2 h. The reaction mixture was partitioned between water and ethyl acetate aided by vigorous stirring and heating. The organic layer was washed with brine, dried over sodium sulfate and concentrated to give 6-(4,5-dihydro-1,3-oxazol-2-yl)-1H-indazole (9-1) as a brown solid. ¹H NMR (300 MHz, CD₃OD) δ 8.11 (br s, 2H), 7.83 (d, 1H, J=8.5 Hz), 7.70 (br d, 1H, J=8.8 Hz), 4.54 (t, 2H, J=9.8 Hz), 4.06 (t, 2H, J=9.8 Hz). LRMS m/z (M+H+CH₃CN) 188.2 found, 188.1 required.

This intermediate was converted to the title compounds by the procedure described in Example 1 Steps 3-8, using morpholine in Step 8.

LRMS m/z (M+H) 402.5 found, 402.2 required

All the above examples gave an IC50 of <1 μM towards BRSK in the HTRF assay. Examples 1 to 9 showed an IC50 of <500 nM towards both BRSK and MARK3 and hence may be regarded as dual BRSK/MARK inhibitors, whereas Examples 10 to 15 showed selectivity for BRSK (IC50_{MARK}>1 μM and/or at least 4-fold greater than IC50_{BRSK}).

1. A screening method for selecting compounds suitable for use in the treatment or prevention of Alzheimer's disease or other condition involving abnormal phosphorylation of tau, said method comprising the steps of:
(i) incubating BRSK with a peptide substrate in the presence of ATP and a test compound, said peptide substrate comprising a phosphorylatable serine residue, under conditions compatible with phosphorylation of said serine residue;
(ii) measuring the degree to which the peptide substrate has become phosphorylated at said serine residue; and
(iii) comparing the result obtained in step (ii) with that obtained from a corresponding blank incubation carried out in the absence of a test compound.

2. The screening method of claim 1 wherein the test compound is further screened for activity against MARK.

3. The screening method according to claim 1 wherein the test compound is further screened for activity against one or more additional kinases selected from Cdk-5, PKA, GSK3β and CaMKII.

4-8. (canceled)

9. A method of treating or preventing Alzheimer's disease or other condition involving abnormal phosphorylation of tau comprising administering to a patient in need thereof a therapeutically-effective dose of a BRSK inhibitor; where the term “BRSK inhibitor” refers to a compound which, when tested in the screening method of claim 1, produces a lowering of the degree of phosphorylation in comparison with that obtained from the blank incubation.

10. The method according to claim 9 wherein said BRSK inhibitor is selective for BRSK over MARK.

11. The method according to claim 9 wherein said BRSK inhibitor is also active against MARK.

12. The method according to claim 9 wherein said BRSK inhibitor is selective for BRSK over one or more additional kinases selected from Cdk-5, PKA, GSK3β and CaMKII.

13. The method according to claim 9 wherein said BRSK inhibitor is a compound of formula 5: 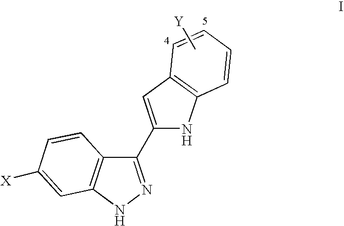
or a pharmaceutically acceptable salt thereof; wherein Y is attached at the 4- or 5-position of the indole ring and X and Y are as indicated in the following table:

| Ex. | X | Y | Y-posn. |
|---|---|---|---|
| 1 | CN | 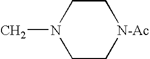 | 5 |
| 2 | CN | 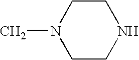 | 5 |
| 3 | CN | CH₂NHCH₂CH₂NH₂ | 5 |
| 4 | CN | 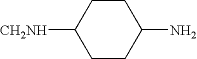 | 5 |
| 5 | CN | 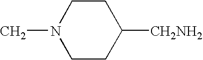 | 5 |
| 6 | CN | 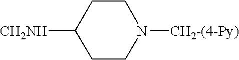 | 4 |
| 7 | CN | 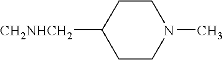 | 4 |
| 8 | CN | (morpholin-4-yl)methyl | 5 |
| 9 | CN | CH₂NHCH₂CH₂—S-t-butyl | 4 |
| 10 | Cl | 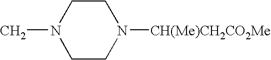 | 4 |
| 11 | 1-H-tetrazol-5-yl | 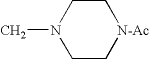 | 5 |
| 12 | 1-H-tetrazol-5-yl | 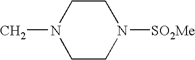 | 5 |
| 13 | CO₂Me | (morpholin-4-yl)methyl | 5 |
| 14 | CONHMe | (morpholin-4-yl)methyl | 5 |
| 15 | 4,5-dihydro-oxazol-2-yl | (morpholin-4-yl)methyl | 5 |
